(19) United States
(12) Patent Application Publication
(10) Pub. No.: US 2004/0181261 A1
(43) Pub. Date: Sep. 16, 2004

(54) Cavernous nerve stimulation device

(76) Inventor: Joseph Manne, New York, NY (US)

Correspondence Address: MUSERLIAN AND LUCAS AND MERCANTI, LLP, 475 PARK AVENUE SOUTH, NEW YORK, NY, 10016, US

(21) Appl. No.: 10/738,707

(22) Filed: Dec. 16, 2003

Related U.S. Application Data
(60) Provisional application No. 60/433,755, filed on Dec. 16, 2002.

(51) Int. Cl. A61N 1/18

(52) U.S. Cl. 607/39

(57) ABSTRACT

Cavernous nerve stimulation is induced by means of a device which creates a time varying magnetic field which, in turn, creates an electric field in a direction parallel to the nerve and at the nerve so as to cause depolarization leading to an action potential and subsequent sensory stimulation and erection.

### CROSS REFERENCE TO RELATED APPLICATIONS

This Application claims the priority under 35 USC 119 of U.S. Provisional Patent Application No. 60/433,755, filed Dec. 16, 2002.

### BACKGROUND OF THE INVENTION

1. Field of the Invention

This invention relates to electrical stimulation of a cavernous nerve and a device for doing the same.

2. Description of the Prior Art

Nerve impulses are transmitted in the body by the nervous system which includes the brain, spinal cord, nerves, ganglia and the receptor. Nerves are made up of axons and cell bodies together with their respective protective and supporting structures. The axon is the long extension of the nerve cell that conducts nerve impulses to the next neuron.

The propagation of the nerve impulse along the axon is associated with an electric potential and a flow of cations into and out of the axon. This electric potential is called the action potential. The typical human action potential has an advancing front of depolarization with a peak value of +40 mV. In order to continue to propagate, the action potential must trigger the depolarization of the neural tissue directly at the front of the advancing wave.

In order to produce depolarization, the interior of the axon must be depolarized from its resting potential of −70 mV (a typical resting voltage potential) to a potential of −60 mV. However, once −60 mV is reached, the sodium channels in the axon opens and causes sodium cations (Na+) to flow into the axon, thereby allowing the depolarization to proceed to +40 mV. Other ion channels then open and cause the potassium cations in the axon to flow out of the axon until the interior of the cell repolarizes to −70 mV.

Thus, all that is necessary to propagate the action potential is to have an external potential which can bring the interior of the cell to −60 mV. Since the action potential consists of an advancing wave of +40 mV, under normal conditions the interior of the cell will depolarize to −30 mV (−70 mV+40 Mv), which is more than enough to propagate the action potential. As is known, these potentials are externally induced transmembrane potentials which are measured across the wall of the axon.

Given the fact that the nerve impulse is transmitted along the axon due to an electrical potential (the action potential), there have been a number of studies into the artificial propagation of nerve impulses using various electrical devices. For example, electrodes have been inserted into a nerve and a current passed through the nerve to cause movement of muscles.

Direct application of electric current has also been used to effect neurostimulation. In this technique, electrodes were applied directly to the skin or to underlying structures in a way which created an electric current between the two electrodes in the tissue in which the target neuronal structure was located. This technique employed a constant voltage source and was intended to cause neuronal transmission and thereby produce stimulation both in peripheral nerves and in the brain.

The influence of an external electric field on neuronal tissue has also been studied. One model for this is the effect of a monopolar electrode in the proximity of a neuron. (Rattay F, J Theor. Biol (1987) 125,339-349). The model for electrical conduction in the neuron, which has been widely accepted, is the modified cable equation:

$$\partial \frac{V}{\partial t} = [ \frac{d}{4 {ρ}_{i}} ( \dot{\partial } \frac{V}{\partial {x}^{2}} + \partial \frac{{V}_{e}}{\partial {x}^{2}} ) - {i}_{i} ] / {c}_{m} \qquad ( eqn 1 )$$

where:

V represents voltage,

iᵢ is the total ionic current density,

ρᵢ=the resistivity of the axoplasm,

cₘ=capacitance of the membrane,

Ve=externally applied voltage,

the term:

$$\partial \frac{{V}_{e}}{\partial {X}^{2}} \qquad ( eqn 2 )$$

is referred to the activating function by Rattay because it is responsible for activating an axon by external electrodes.

The activating function has two possible effects on an axon. If its magnitude is sufficient there is a superthreshold response. This leads to the generation of an action potential. If this occurs then the cable equation will predict the expected response. In order to calculate the equation however the ionic current term iᵢ must be calculated.

In order to calculate the ionic current, an equation of membrane ionic current, as a function of the externally induced transmembrane potential, is used. For myelinated membranes the Hodgkin-Huxley equation can be used. For unmyelinated membranes the Huxley-Frankenhaeuser equation is used.

There are other equations which account for membrane temperature as well. The other possible effect on the axon is subthreshold stimulation.

If a subthreshold stimulus is applied, then the transmembrane voltage is directly related to the activating function. The voltage changes due the opening of voltage sensitive ionic channels can be ignored. The calculation of transmembrane voltage becomes simplified. It is the subthreshold stimulation of the neuron which is considered in this patent application.

### SUMMARY OF THE INVENTION

The present invention stimulates a cavernous nerve of a patient by exposing the cavernous nerve of the patient to different spatially and temporally varying electromagnetic fields by means of a magnetic flux generator positioned external to the patient close to the cavernous nerve. More specifically, the invention generates an electric current in the cavernous nerve producing areas of depolarization which lead to the propagation of nerve signals in the cavernous nerve. Thus, this invention provides for an entirely new form of stimulation both sensory stimulation and motor (erectile function) stimulation.

The term cavernous nerve as used herein means one or more nerves which have erectile function in an animal, especially, a human being.

This invention also provides a localizing system. In the prior art of neurostimulation, an electronic device is usually implanted in the patient. However, with implanted electronic devices, complexity and cost increases. The present invention avoids these problems. Broadly, the method of the present invention comprises:

(1) creating a magnetic flux with a magnetic flux generator, said generator being positioned completely external to the patient; and

(2) treating a cavernous nerve of said patient with said magnetic flux to cause an electric current along an axon which leads to a focused propagation of an action potential in said cavernous nerve thereby resulting in an erection.

As is known, a time varying magnetic field, which is a magnetic flux, results in an electric field. The orientation and strength of the magnetic flux and its resulting electric field is such that it depolarizes regions along the axon so as create areas of neurostimulation. Electric current is thus caused to travel in the cavernous nerve.

To accomplish stimulation in accordance with the present invention, a preliminary study is performed wherein the nerve response to specific stimuli is recorded. More specific microelectrodes are used to measure the individual axonal response of a nerve to specific stimuli. The axonal action potentials created by the magnetic flux reproduce the previously measured axonal responses to stimuli thereby causing an erection and sensory stimulation in the brain.

The strength of depolarization is expressed as a voltage and is a measurement of the voltage or electric potential between the inside of the axon and the outside of the axon. This electric potential is sometimes referred to as the externally induced transmembrane potential since it is measured across the cell wall.

For depolarization, the magnetic flux should be of such an orientation and strength so as to create a net externally induced transmembrane potential equal to or greater than −60 mV. More preferably, the electric potential created by the magnetic flux of the present invention should be about −50 mV or greater and, more preferred, about −40 mV or greater. It should be understood that “greater” means more positive.

The orientation of the electric field is such that it has a component parallel to the long axis of the axon.

Preferably, the configuration of magnetic flux generators produces a depolarized region. This ensures propagation of a nerve signal down an axon.

The magnetic flux generator of the present invention is a RLC (resistor, inductor, capacitor) circuit with a coil of wire as the inductor. The magnetic flux generates a depolarized region. The externally induced transmembrane potentials for the depolarized region have a strength and orientation as referred to above, e.g. depolarized equal to or greater than −60 mV.

Suitable, the coil of wire can have various shapes and sized such as round, figure eight, square, torroidal, etc. Most preferably, the magnetic flux generator is an RLC circuit with a round coil of wire. As will be appreciated by one of skill in the art, any high DC voltage pulsed power supply can be employed with the inductive elements.

The round coil of wire is preferably in series with a capacitor and a resistor so as to form a RLC circuit. The capacitor is discharged through the device so as to form a time varying magnetic field which in turn creates an electric field.

Furthermore, a plurality of magnetic flux generators can be used such that no one individual magnetic flux generator produces the necessary electric field but that the combined generators, when oriented towards the cavernous nerve to produce a net electric field. That net electric field is of sufficient magnitude to produce a depolarized region of sufficient externally induced transmembrane potential to produce a unidirectional neural impulse.

The round coil of wire is preferably housed within a housing so as to insulate the round coil of wire from the skin. Examples of a preferred housing is a ring, a patch, or a prophylactic. The housing is adapted to be worn on the outside of the penis so as to maintain the coil in close proximity to the cavernous nerve during movement of the cavernous nerve.

The round coil of wire is designed to be mounted in a plane substantially perpendicular or substantially parallel to a plane of the cavernous nerve. This substantially parallel arrangement would also include a half-wrap around the penis. All of these configurations may be incorporated into a housing as well.

The capacitor and the voltage source are mounted external to the housing that houses the coil. Suitably, they are located in a pouch that is worn by the patient, or in a pouch that is beside the patient.

Broadly, the present invention can be described as follows:

A method for stimulating the cavernous nerve in a patient comprising the steps of:

creating a time varying magnetic field with a device positioned completely external to said patient, said time varying magnetic field resulting in an electric current in said cavernous nerve, for as long as required, said electric current causing the progation of a neural impulse in said neural tissue thereby resulting in an erection.

Preferably, the device comprises a coil of wire which can produce a time varying magnetic field.

More preferably, said coil of wire is housed within a housing so as to insulate said coil of wire.

Suitably, said device comprises a resistor, a capacitor and a coil of wire in series.

More preferably, said capacitor is discharged through said coil of wire so as to form a time varying magnetic field which in turn creates said electric field.

More preferably, said coil of wire is circular in shape and has about 7 to about 10 turns, and said coil of wire has a diameter of about 3 to about 7 cm.

Preferably, a time varying current passes through said device and said time varying current increases from 0 to about 6000 amps in 60 microseconds.

More preferably, said coil of wire has a resistance (R) of about 0.1 to about 0.5 ohms and a inductance (L) of about 10 to about 90 microhenries.

Alternatively, the present Invention can be described as follows:

An electrical device for producing a nerve impulse in the cavernous nerve so as to produce an erection, said device comprising:

a RLC circuit comprising a resistor, a coil of wire, and a capacitor, wherein said coil of wire is positioned so as to situate said coil in close proximity to said cavernous nerve;

a housing for insulating said coil of wire.

Preferably, said housing is a ring, a patch, or a prophylactic; and said housing is adapted to be worn on the outside of a penis.

Suitably, said coil of wire completely encircles a penis.

Preferably, said coil of wire is in a plane substantially perpendicular to a plane of said cavernous nerve.

Suitably, said coil of wire is in a plane substantially parallel to a plane of said cavernous nerve.

Preferably, said coil of wire is half-wrapped around a penis.

Preferably, current produces an electrical field which creates a triphasic potential within the cavernous nerve, said triphasic potential consisting of a virtual cathode surrounded by a virtual anode on either side of said virtual cathode.

Suitably, the RLC circuit creates a time varying magnetic field, said time varying magnetic field resulting in an electric current in said cavernous nerve, for as long as required, said electric current causing the progation of a neural impulse in said neural tissue thereby resulting in an erection.

Suitably, said coil of wire is circular in shape and has about 7 to about 10 turns, and the coil has a diameter of about 3 to about 7 cm.

Preferably, a time varying current passes through said RLC circuit and said time varying current increases from 0 to about 6000 amps in 60 microseconds.

Preferably, said coil of wire has a resistance (R) of about 0.1 to about 0.5 ohms and an inductance (L) of about 10 to about 90 microhenries.

### BRIEF DESCRIPTION OF THE DRAWINGS

These and other aspects of the present invention may be more fully understood by reference to one or more of the following drawings wherein:

FIG. 1 is an overall schematic of the present invention illustrating the magnetic flux generator as a round coil in a RLC circuit;

FIG. 2 illustrates a nerve modeled as a lumped circuit;

FIG. 3 illustrates a computer program used to calculate externally induced transmembrane potentials;

FIG. 4a illustrates the calculated electric field along a length of axon;

FIG. 4b illustrates the calculated externally induced transmembrane potential along a length of axon using the computer program;

FIGS. 4c-4g show the graphs of the externally induced transmembrane potential across different cross sections of the nerve bundle, corresponding to FIG. 4b;

FIG. 4h illustrates the calculated externally induced transmembrane potential along a length of axon using the computer program with a different set electrical circuit parameters than FIG. 4b;

FIGS. 4i-4k show the graphs of the externally induced transmembrane potential across different cross sections of the nerve bundle, corresponding to FIG. 4h;

FIG. 5 illustrates current versus time for a coil used in accordance with the present invention;

FIG. 6 illustrates two proximate coils used to maintain the magnetic flux;

FIGS. 7a-7i illustrate a several arrangements of the coil of the present invention; and

FIG. 8 illustrates a schematic for a circuit that can deliver high frequency, high voltage DC current.

### DETAILED DESCRIPTION OF THE INVENTION

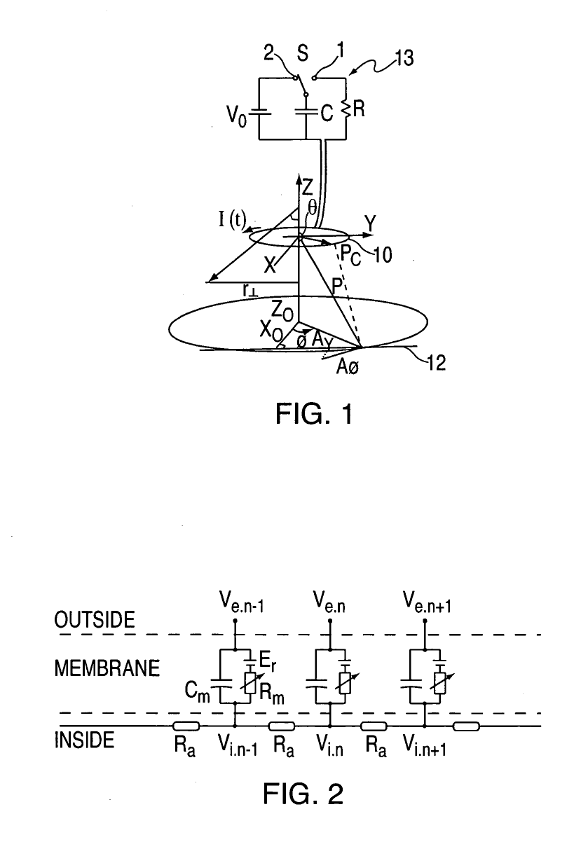

FIG. 1 illustrates magnetic coil 10 overlying axon 12 and the coordinate system used to describe the three dimensional space around these elements. Coil 10 which lies above axon 12 consists of a series of turns of a conduction wire typically copper although other metals or alloys can be used. Coil 10 is the inductive component in the RLC circuit 13.

RLC circuit 13 is well known to those experienced in the art of electronics. It consists of resistor R, inductive element L, which is coil 10 in the circuit shown in FIG. 1, and capacitor C in series. The behavior of the RLC circuit 13 is well known to those experienced in the field of electronics. Voltage source Vo in conjunction with switch So is used to charge capacitor C when switch S is in position 1.

Capacitor C is then discharged through resistor R and coil 10 when switch S is moved to position 2. The total current I(+) and the rate of change of the current when capacitor C is discharged through resistor R and coil 10 can be calculated in a conventional manner.

Axon 12 is parallel to the y-axis; it lies Zₒ below the coil and Xₒ from its axis. When capacitor C is discharged, the current, I(t) in the coil induces an electric field in the tissue whose gradient in the direction of the nerve axis is the activating function. It determines the local transmembrane current in the axon and is related to the magnetic vector potential, Ao, and its component lying along the axon, Ay.

The externally induced transmembrane potential in axon 12 is calculated using the cable equation. The cable equation is:

$$\begin{matrix}{δ}^{2} \frac{{\partial }^{2} V}{\partial {x}^{2}} - V - α \frac{\partial V}{\partial t} = {δ}^{2} \frac{\partial {E}_{x}}{\partial x} & ( eqn 3 ) \\ \frac{\partial {E}_{x}}{\partial x} & ( eqn 4 )\end{matrix}$$

where

δ=space constant of the cable equation,

α=the time constant of the cable equation, the term:

$$E = - Δ ϕ - \frac{1}{c} \frac{\partial A}{\partial t} \qquad ( eqn 5 )$$

is referred to as the activating function. The activating function determines the transmembrane voltage for subthreshold stimulation.

One of Maxwell's Equations relates the electric field E along the axon to the magnetic potential A created by the coil 10: where the first term in the equation:

−Δφ

represents the contribution to the electric field from fixed charge. In this particular example, there is no fixed charge contributing to the field thus this term can be eliminated and the equation for the electric field becomes:

$$E = - \frac{1}{c} \frac{\partial A}{\partial t} \qquad ( eqn 6 )$$

For the case of the coil with a time varying current, such as coil 10 in the present invention, the equation for the induced magnetic vector potential has been described (Jackson J D. Classical Electrodynamics. 1962, New York):

$$\frac{A}{c} = I ( t ) \frac{{ρ}_{c} μ}{π} {\int }_{0}^{2} \frac{π cos φ ⅆ φ}{\sqrt{{( ρ c )}^{2} + {ρ}^{2} - 2 ρ {ρ}_{c} sin θ cos φ}} \qquad ( eqn 7 )$$

the equation simplifies to:

$$\frac{A}{c} = I ( t ) \frac{{ρ}_{c} μ}{π \sqrt{{( ρ c )}^{2} + {ρ}^{2} - 2 ρ {ρ}_{c} sin θ cos φ}} \times \\ ( \frac{2}{{k}^{2}} ( ( K ( k ) - E ( k ) ) - K ( k ) ) ) \qquad ( eqn 8 )$$

where k is defined by:

$${k}^{2} = \frac{4 {ρ}_{c} ρ sin θ}{{ρ}_{c}^{2} + {ρ}^{2} - 2 ρ {ρ}_{c} sin θ} \qquad ( eqn 9 )$$

substituting the expression for the magnetic vector potential into the expression for the component of the electric field along the axon 12 (y component):

$$\begin{matrix}E = ( \frac{\partial A}{\partial t} ) \\ = \frac{ⅆ ( I ( t ) )}{ⅆ t} \frac{{ρ}_{c} μ cos ( φ )}{π \sqrt{{( ρ c )}^{2} + {ρ}^{2} - 2 ρ {ρ}_{c} sin θ cos φ}} \times \\ ( \frac{2}{{k}^{2}} ( ( K ( k ) - E ( k ) ) - K ( k ) ) )\end{matrix} \qquad ( eqn 10 )$$

In order to calculate the value of the preceding equation, the time derivative of the current (dI/dt) must be determined. But to do this an expression for the current in the coil 10 must be obtained.

The circuit for the magnetic coil takes the form of an RLC circuit. There is a resistor R, a capacitor C, and the coil 10, which is the inductive element (L). The inductance of the coil can be calculated by standard formulas known to those experienced in the art (Smythe W R Static and Dynamic Electricity. New York: McGraw-Hill, 1968):

$$L = {μ}_{0} {r}_{c} {N}^{2} ( ln ( 8 \frac{{r}_{c}}{{r}_{w}} - 1.75 ) ) \qquad ( eqn 11 )$$

where

L is the inductance of the coil,

rc is the coil radius,

rw is the wire radius,

N is the number of turns in the coil, and

mu is the magnetic permeability.

The equation for the current in an underdamped RLC circuit is also well known to those experienced in the art of electronics:

$$I ( t ) = \frac{{V}_{o}}{{w}_{b} L} {ⅇ}^{( - {w}_{a} δ ) t} sin {w}_{b} t \qquad ( eqn 12 )$$

where

wₐ=(1/LC)^{0.5}

w_{b}=wₐ(1−delta²)^{0.5}

δ=R/2(C/L)^{0.5}

Vo=initial voltage across capacitor,

C is the capacitance of the capacitor (in farads), and

R is the resistance of the resistor (in ohms).

The axial electric field gradient is the source term in the modified cable equation (Equation 1) for nerve conduction. The ultimate objective is to calculate the other variable in the equation which is the externally induced transmembrane potential, V in Equation 3.

Equation 3 is a nonlinear second order partial differential equation. Traditionally all equations in this class tend to be difficult to solve analytically. There have been many publications describing the solution of the modified cable equation (Nagarajan S S, Durand D M and Warman E N. Effects of Induced Electric Fields on Finite Neuronal Structures: A Simulation Study. Transactions on Biomedical Engineering 40(11), pgs;1175-1187), 1993).

It has been a common practice to reduce the equation to a series of linear differential equations using a compartmental analysis technique. (Segev I Fleshman W and Burke RE, “Compartmental Models of Complex Neurons” Methods of Neuronal Modelling: From Synapses to Networks, Koch C and Segev I, Eds. Cambridge, Mass.; MIT Press, 1989, pgs:63-97)

In this method the nerve is divided into N compartments. Each compartment is modeled as a lumped circuit. The repeating unit of this compartmental circuit is shown in FIG. 2. In the case of a myelinated nerve the repeating unit can be taken as the portion the nerve bounded by two adjacent nodes.

The axial current in each compartment is secondary to two factors. The first is the voltage gradient along the axon. The second is the extrinsically induced electric field from the externally fluxing magnetic field. The current can be related to these electric potential terms by Ohm's Law in the following fashion:

I=G(Vₐ−V_{b})−G∫ₐ^{b}Eₓdx (eqn 13)

where a and b are two adjacent nodes,

Iₙₑₜ is the axial current,

G is the conductance in the axon,

Ex is the axial component of the magnetically induced electric field, and

Va and Vb are the voltages at the two adjacent nodes.

In order to extend Equation 13 to the entire axon it is necessary to apply the above equation to a node and its two adjacent nodes such that a current balance equation for the central node is created, The equation for the transmembrane current at the middle node is:

Iₙₑₜ=G(V_{c}−2V_{b}+V_{c})−G(∫_{b}^{c}(Ex)dx−∫ₐ^{b}(Ex)dx) (eqn 14)

where the naming conventions used in Equation 13 apply and where Vb is the potential in the center node and Va and Vc are the potentials in the two surrounding nodes. The net transmembrane current can be expressed as the sum of channel current and the current due to the capacitative elements in the cell:

$${I}_{t} = C \frac{ⅆ v}{ⅆ t} + {I}_{t} \qquad ( eqn 15 )$$

where:

C dV/dt is the capacitative current term, and

I_{ch} is the ionic channel term.

This can be substituted into Equation 14 for the net current term Inet.

In the steady state condition the time dependent terms vanish and this equation now becomes:

I_{ch}=G(V_{c}−2V_{b}+V_{c})−G(∫_{b}^{c}(Ex)dx−∫ₐ^{b}(Ex)dx) (eqn 16)

Finally the transmembrane current through the center node in the subthreshold steady state I_{ch} can be expressed as the product of the channel conductance Gm and the externally induced transmembrane potential Vb.

VₕG_{M}=Gₐ(V_{c}−2V_{b}+V_{c})−Gₐ(∫_{b}^{c}(Ex)dx−∫ₐ^{b}(Ex)dx) (eqn 17)

rearranging terms:

$${V}_{c} - ( ( \frac{{G}_{m}}{{G}_{a}} + 2 ) {V}_{b} ) + {V}_{a} = {\int }_{b}^{c} ( Ex ) ⅆ x - {\int }_{a}^{b} ( Ex ) ⅆ x \qquad ( eqn 18 )$$

Finally the equation is applied to all nodes such that each node is successively treated as the center node with the exception of the two terminal nodes:

$${V}_{n - 1} - ( ( \frac{{G}_{m}}{{G}_{a}} + 2 ) {V}_{n} ) + {V}_{n - 1} = {\int }_{( n ) ⅆ l}^{( n - 1 ) ⅆ l} ( Ex ) ⅆ x - {\int }_{( n - 1 ) ⅆ l}^{( n ) ⅆ l} ( Ex ) ⅆ x \qquad ( eqn 19 )$$

where:

n=2, 3, 4, . . . L−1,

dl is he internodal distance, and

L is the total number of nodes in the nerve segment of interest. For the two terminal nodes the applicable equations are:

$$\begin{matrix}for n = 1 ( ( \frac{{G}_{m}}{{G}_{a}} + 1 ) {V}_{1} ) - {V}_{2} = {\int }_{0}^{2 dl} ( Ex ) ⅆ x & ( eqn 20 ) \\ for n = L {V}_{n} - ( ( \frac{{G}_{m}}{{G}_{a}} - 1 ) {V}_{n - 1} ) = {\int }_{( L - 1 ) dl}^{( L ) dl} ( Ex ) ⅆ x & ( eqn 21 )\end{matrix}$$

Thus, there are L equations in L unknowns. The unknowns are the externally induced transmembrane potentials located in the vector V=(V1,V2,V3, . . . V1). The known quantities in the equations are the internodal potential differences due to the externally induced electric field: E={E1,E2,E3,E4 . . . EL}. These are L linear simultaneous equations which can easily be solved by a variety of techniques.

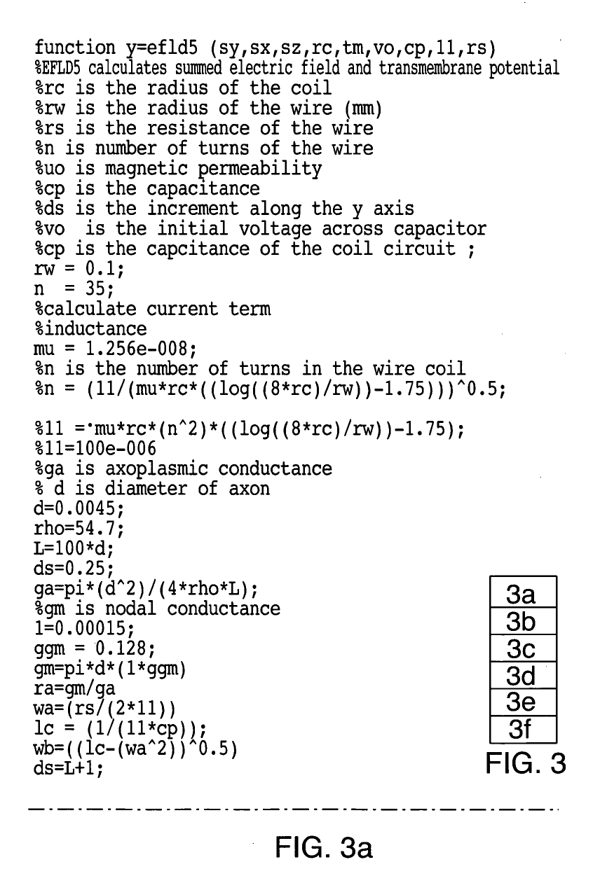

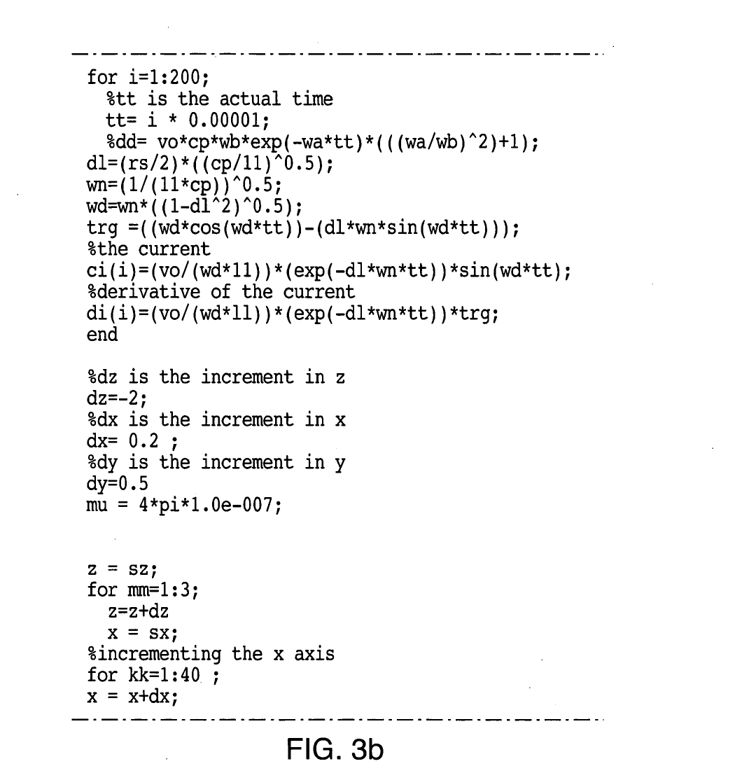

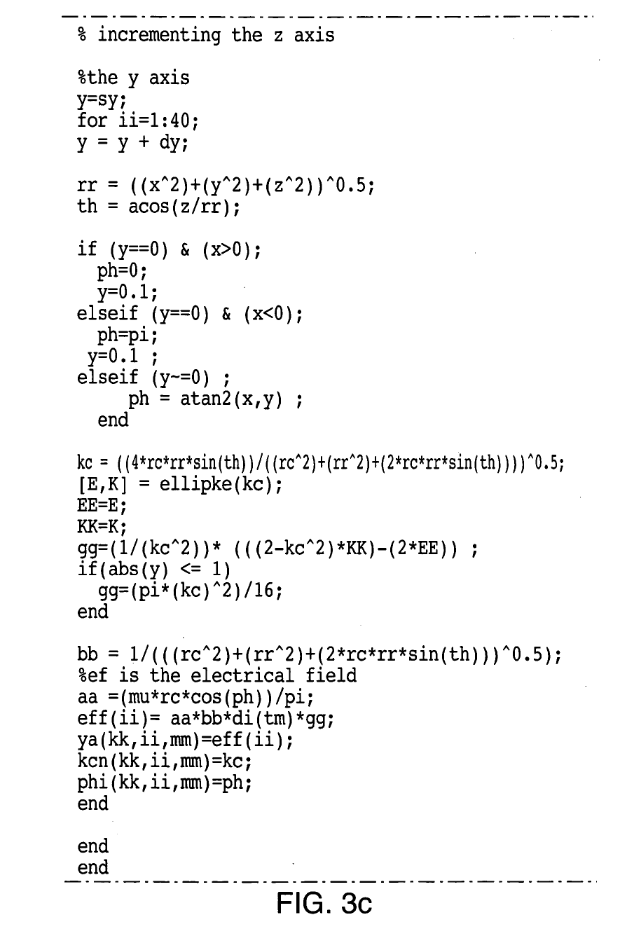

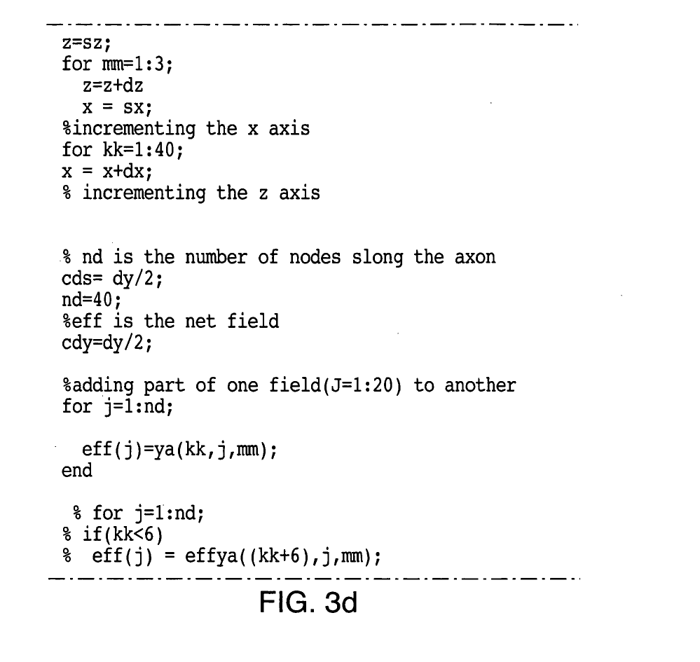

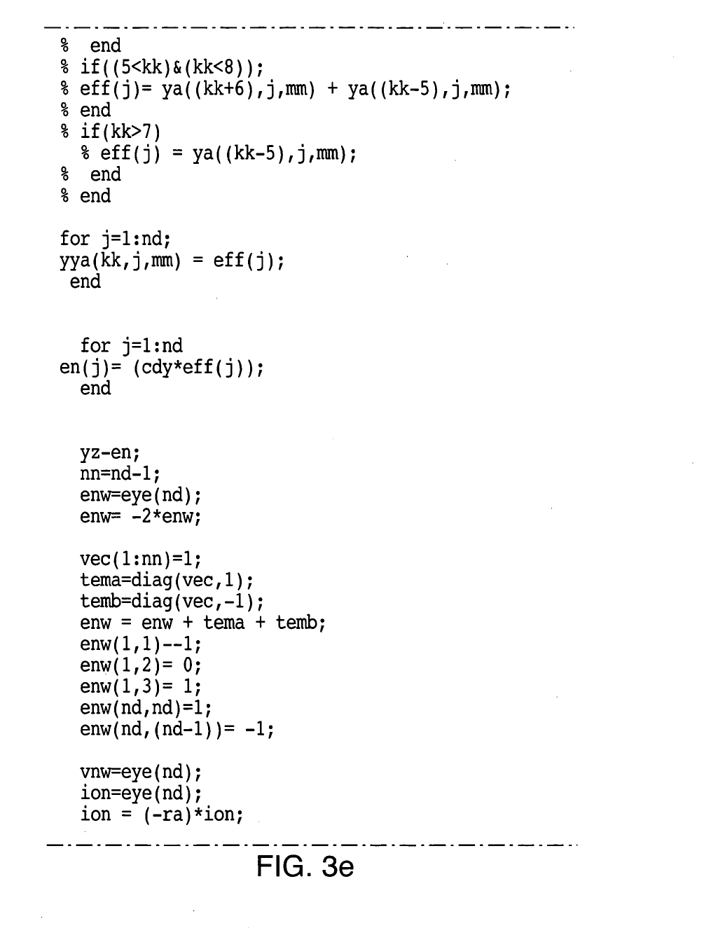

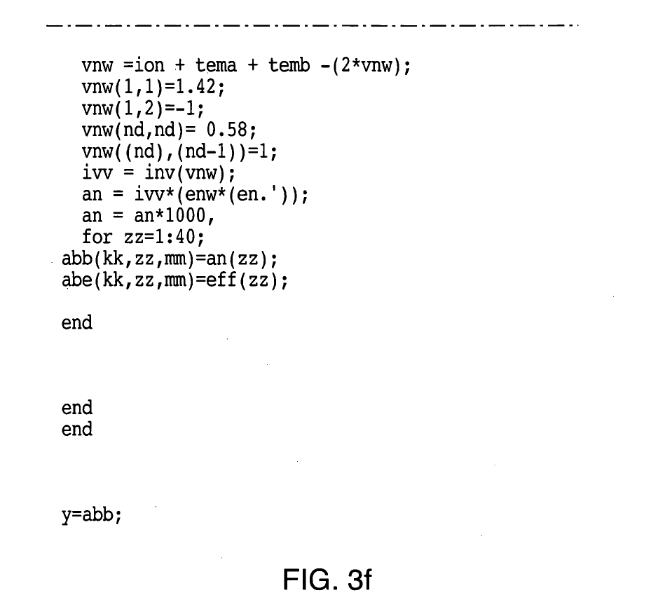

A computer program, shown in FIG. 3, calculates the externally induced transmembrane potentials in the subthreshold condition. In this program the simultaneous equations are expressed as the matrix product of the matrix: A which contains the coefficients for V1,V2 . . . and the product of the matrix: B which contains the coefficients of E1,E2,E3 . . . leading to the following equation:

A*V=B*E (eqn 22)

Then V can be solved for:

V=A⁻¹*B*E (eqn 23)

Using the preceding equations it is possible to calculate the correct coil and circuit parameters to produce a region of depolarization along the nerve. Depolarization leads to a propagating neural impulse.

Thus, a neural impulse can be propagated in a unidirectional fashion. The typical human action potential has an advancing front of depolarization with a peak value of 40 mv. In order to continue to propagate, the action potential must trigger the depolarization of the neural tissue directly at the front of the advancing wave.

In order to produce depolarization the interior of the axon must be depolarized from its resting potential of −70 mv to a potential of −60 mv. Once −60 mv is reached the sodium channels in the cell opens allowing the depolarization to proceed. Other ion channels can then open until the interior of the cell depolarizes to 40 mv.

Thus, all that is necessary to propagate the action potential is to have an external potential which can bring the interior of the cell to −60 mv. Since the action potential consists of an advancing wave of 40 mv, under normal conditions the interior of the cell will be depolarized to −30 mv, which is more than enough to propagate the action potential.

Equations 12, 13, 20, 21, 22, and 23 can be used to calculate the resultant externally induced transmembrane potential induced for a specific set of coil and RLC circuit characteristics. Thus the circuit parameters required to produce +10 mv of depolarization at any one point along an axon can be determined. This is the requirement for producing a unidirectional neural signal.

Equations 12, 13, 20, 21, 22, and 23 were incorporated into the computer program shown in FIG. 3. As described earlier, it calculates the externally induced transmembrane potential when the coil and circuit characteristics are provided. The input (independent variables) are coil radius, resistance capacitance, inductance and initial voltage for the RLC circuit, and the position of the coil with respect to the target axon.

As is understood by those skilled in the art, temperature can change the resting potential of the cell and, hence, can cause a change in the necessary electric field for propagation or blocking of the action potential. Additionally, other metabolic conditions can cause a change in the resting potential and, hence, would require a change in the necessary electric field generated by the magnetic flux in accordance with the present invention. Furthermore, it is known that some nerves have resting potentials which are greater than −70 mv. Again, adjustments are made to the magnitude of the magnetic flux so as to produce the necessary externally induced transmembrane potential.

### EXAMPLE 1

This example illustrates stimulation in accordance with the present invention.

Using this computer program it is thus possible to calculate the correct values for the RLC circuit and coil to produce sufficient transmembrane voltage to produce a unidirectional focused neural impulse. There are different possible combinations of circuit and coil parameters which will satisfy the required transmembrane voltage criteria. The following example illustrates one set of conditions which produce the desired effect. One set of values of circuit and coil which yield sufficient externally induced transmembrane potentials are:

Rc=radius of coil=1 cm,

Rw=radius of wire=1 mm,

Resistance R=0.21 ohms,

Capacitance C=0.0072 Farad,

Inductance of coil=L=9e-5 Henry,

Voltage=Vo=700 volts, and

z0=height is coil above axon=3 cm.

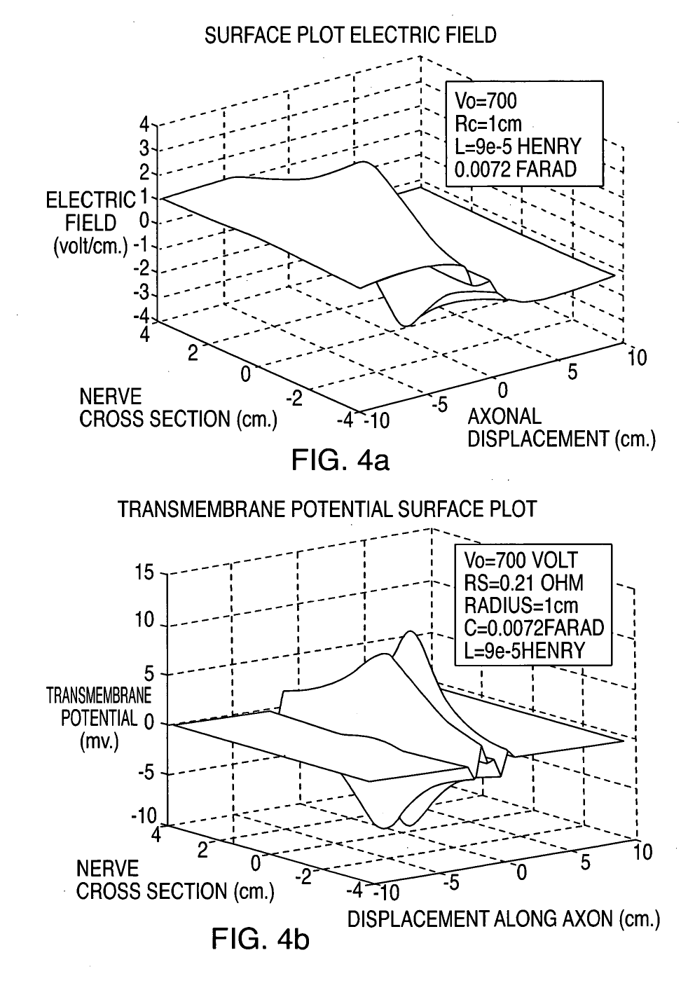

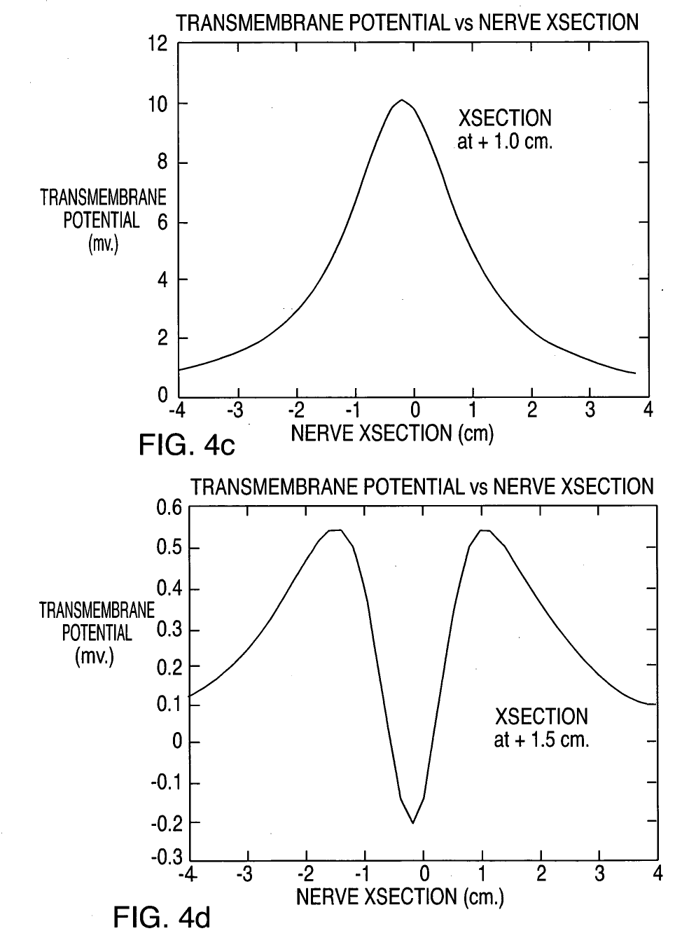

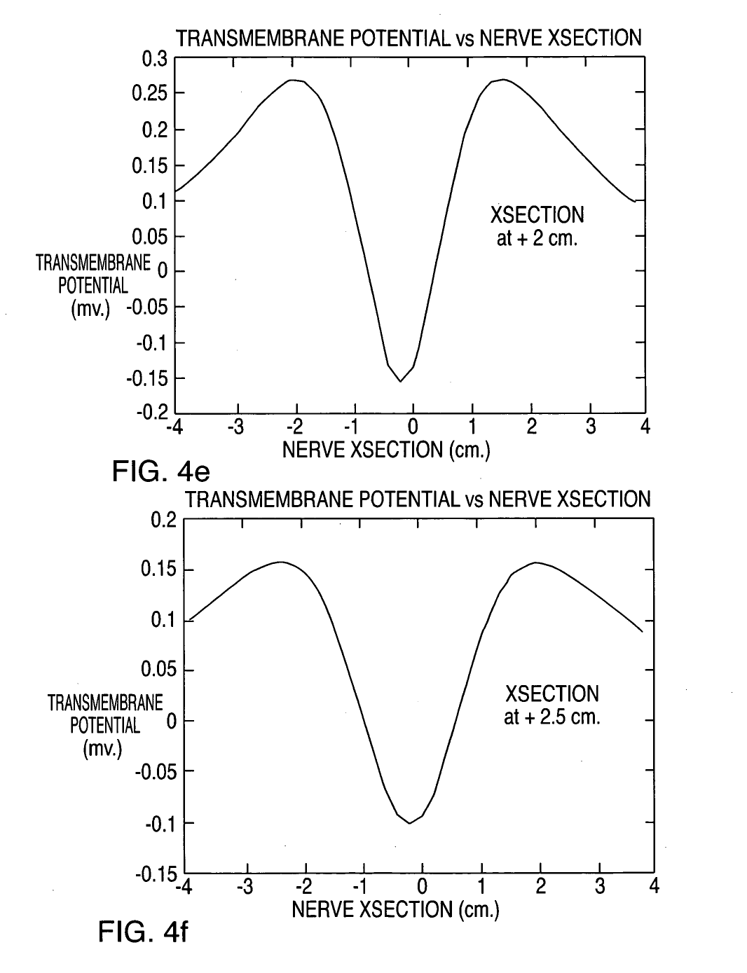

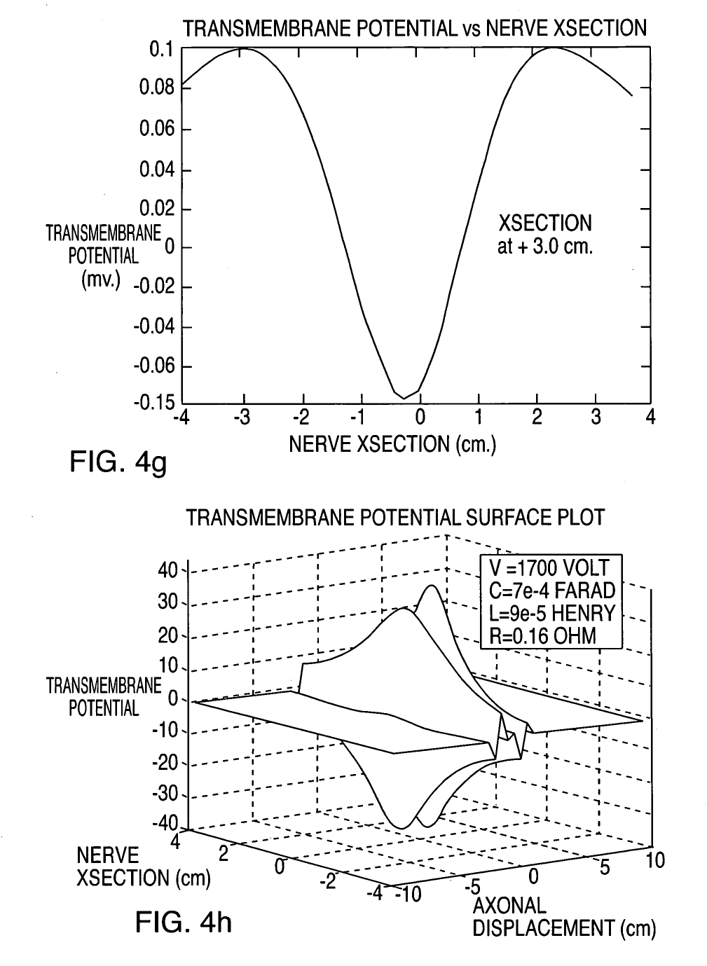

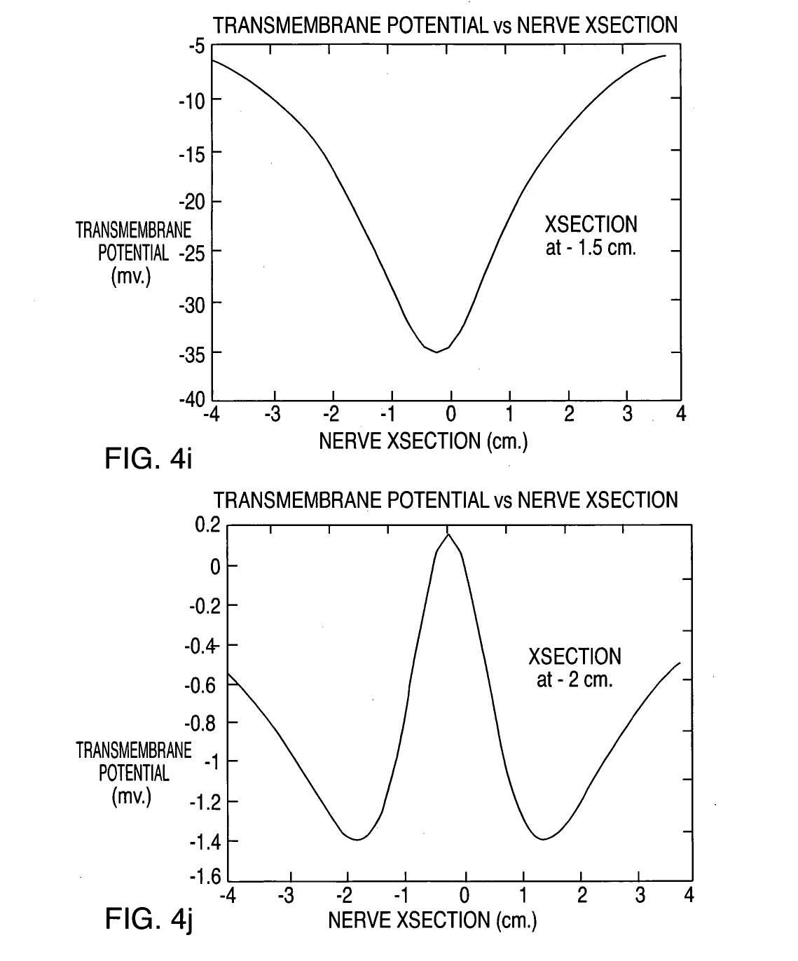

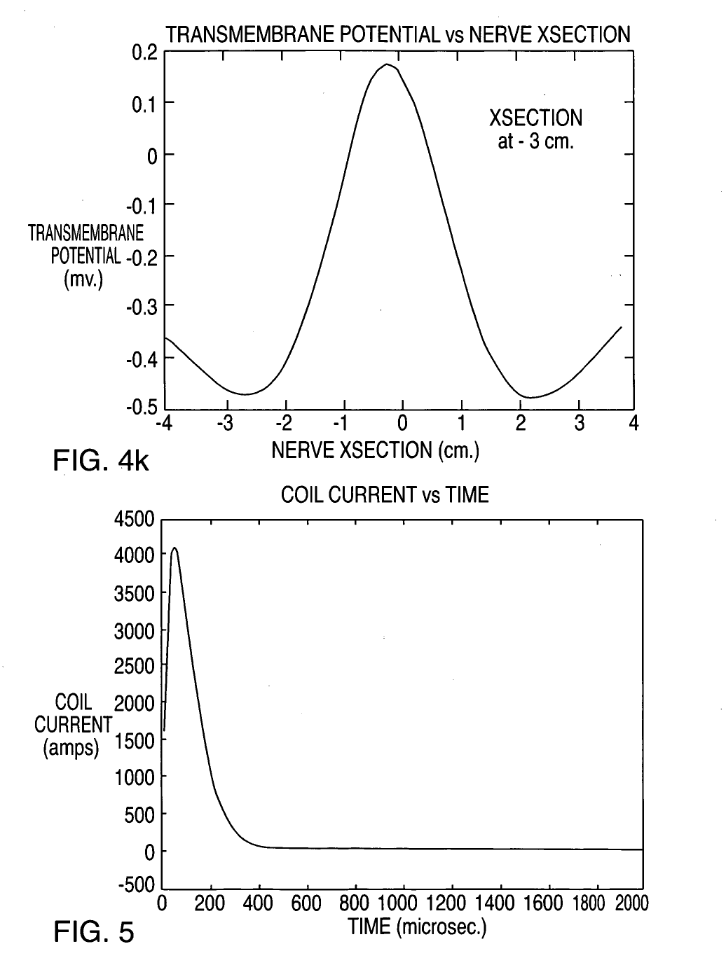

FIGS. 4a and 4b show the results of the calculation of induced electric field and externally induced transmembrane potential, respectively, along the length of the axon using the preceding coil and circuit values. Note that this is the externally induced transmembrane potential induced by the coil. The net transmembrane potential would be equal to the induced transmembrane potential plus the resting potential of the neuron (e.g. −70).

The time is 60 microseconds from the application of 100 volts dc across the circuit. FIG. 4a shows a surface plot of the induced electrical field in neuronal tissue in a plane 3 cm below the coil. The x and y axes represent all points in that plane. The vertical z axis represents the electric field strength in FIG. 4a and the externally induced transmembrane potential (millivolts) in FIG. 4b.

From examination of the graph in FIG. 4a, it can be seen that there is a maxima and a minima in the electric field. Similarly there are multiple maxima and minima in externally induced transmembrane potential as shown in FIG. 4b. The minima correspond to hyperpolarized points. To block propagation of neural impulses these points have to have an induced transmembrane potential more negative than −30 mv. In that case the net transmembrane potential at these points will be greater than −100 mv and thus cannot be depolarized by an advancing action potential.

Among the maxima in the graph there are points with an induced transmembrane potential of greater than 10 mv. Thus the net transmembrane potential at these points will be greater than −60 mv and thus can initiate depolarization and an axonal impulse.

FIGS. 4c-4g show the graphs of the externally induced transmembrane potential across a specific cross section of the nerve bundle. These graphs correspond to the coil used for FIGS. 4a and 4b. FIGS. 4c-4g would correspond to a specific y value (position along the axon). The vertical axis shows the externally induced transmembrane potential at each point along a cross section of a nerve bundle. The calculation was done for a cross section from x=−4 to +4 cm.

Obviously there are no nerve bundles with such a large diameter. However the graph allows one to see how the externally induced transmembrane potential is affected by the positions of a nerve bundle with respect of the overlying coil. The electrical parameters for the coil circuit are those given in Example 1. The data presented in these graphs is simply a subset of the data shown in FIGS. 4a and 4b.

FIG. 4c shows the externally induced transmembrane potential at the nerve bundle cross section which is +1.0 cm from the center of the coil. At this point it can be seen that there is a sharply delineated zone where the externally induced transmembrane potential rises to a value of 10.2 mv. This is just above the value necessary to cause depolarization. What is even more important is the singularity of the maxima and the linearity of the data around this point.

These two features provide for the stimulation of only a small zone within a nerve bundle. Ideally, a single axon within the bundle is stimulated. One of the critical objects of this invention is the ability to cause focal axonal stimulation.

That is because different axonal fibers within a nerve bundle correspond to different end organ receptors and thus would be carrying different neural impulses to the brain. In order to accomplish this it is necessary to have a means of creating a focal change in the externally induced transmembrane potential that would not affect adjacent axonal fibers.

Given that the maxima in FIG. 4c is a single point it is possible to make the zone of stimulation as narrow as desired. This can be proven on the basis of the theory in mathematical analysis called the continuity theory. This states that for a curve for any two chosen points (such as the points where the externally induced transmembrane potential equals 9.5 and top of the curve where it is 10.2 mv), all intermediate values on the curve exist.

In fact, it is possible to make minor changes to the voltage so that the maxima can take on values as close to 10.0 as desired. As the maxima get closer and closer to 10 mv, the range of x values (along the cross section of the nerve) for which the externally induced transmembrane potential is greater than 10 mV., can be made as small as desired. This is a result of the aforementioned continuity theory. Thus, it is literally possible to make the zone of axonal stimulation as narrow as desired. Thus, the present invention has the complete ability to focus the stimulation and stimulate only one axon if desired.

In addition, it will be noted that at all cross sectional segments to the right, (more positive than the current one (x=1.0 cm) have no areas of critical depolarization (>10). This can be seen by observing FIGS. 4d (x=1.5 cm), 4e (x=2 cm), 4f (2.5 cm), and 4g (3.0 cm). For the purpose of sensory stimulation, the coil will be oriented so that the region to the right of the coil (x>0) is lying on the proximal side of the neuron (closer to the brain than the coil).

Thus, the present invention provides a means of selectively stimulating a tiny (axonal) segment of a nerve bundle and then propagating that signal. Because of the absence of areas of critical depolarization to the right of the signal origin, it will always remain as focused desired.

As noted before, a conventional preliminary study has to be carried out so as to pinpoint which axon to stimulate for a specific sensory perception. Then this axon is stimulated in accordance with the present invention.

Referring back to FIG. 5, it can be seen that this time interval corresponds to the portion of the current versus time curve where there is a rapid rise in current from 0 amps to 5827.6 amps over a 60 microsecond interval. Therefore, in order to maintain the proscribed transmembrane potential, it is necessary to have an electrical circuit which can maintain this rapid rise of current change through the magnetic coil at all times. In order to accomplish this, two components are needed.

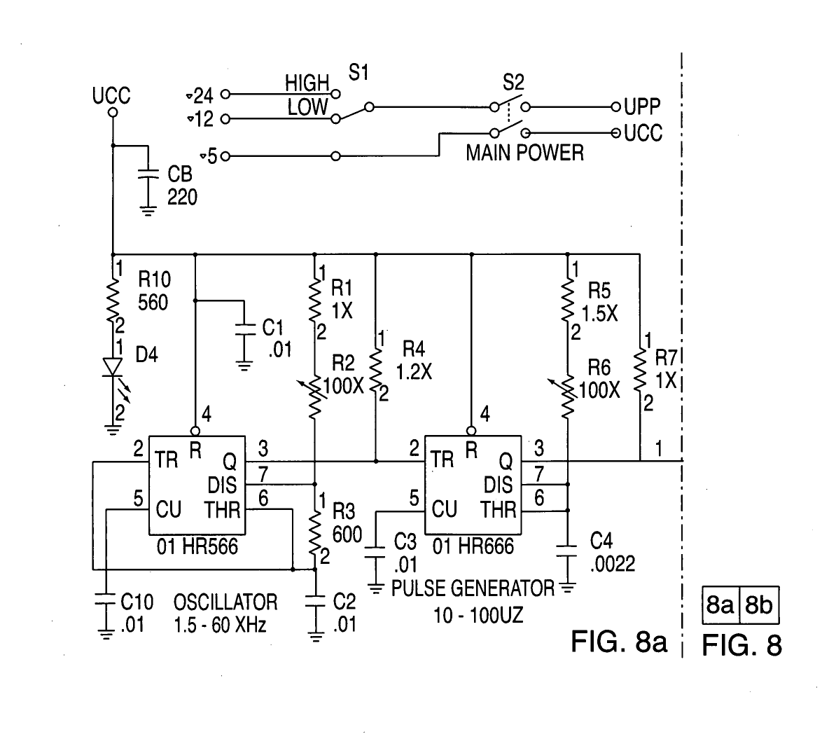

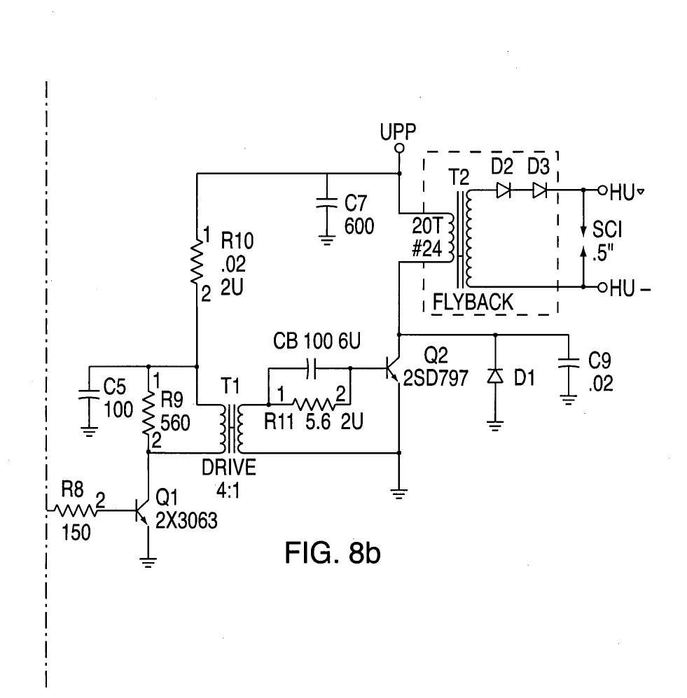

The first is a high voltage DC generator which can produce high voltage DC pulses at a rapid rate. This type of circuit is well known to those experienced in the art of electronics. There are many commercial firms which manufacture such high voltage supplies. One such company is Huettinger Electronic, Inc, (111 Hyde Road, Framington, Conn. 06032, USA). Many other circuits can also be used to produce high frequency, high voltage DC pulses. One such circuit is shown in FIG. 8.

As shown in FIG. 8, horizontal drive transformer (T1) was from small B/W monitor, flyback transformer (T2) was from MacIntosh Plus computer monitor, original primary windings were removed, component values are not critical, output may exceed 25,000 V at certain frequencies with 24 V power—could destroy flyback. Sparkgap provides more protection. Input power was current limited to about 5A. Good heat sink is important on Q2 for continuous operation.

Regardless of how fast the high voltage generator can pulse a RLC circuit, there will be a discontinuity in the current gradient and thus the externally induced transmembrane potential. That is because the current flow in the circuit must drop back down to zero so that there can be another steep rise in current, which is necessary to generate the proper externally induced transmembrane potential.

In order to prevent discontinuity in the externally induced transmembrane potential, there can be two separate RLC circuits with identical values for resistance, inductance, capacitance and initial voltage. Both circuits are powered by high frequency high voltage DC pulses. The two coils are proximate to each other so that they produce the same spatial and temporal distribution of electric fields for a given coil current.

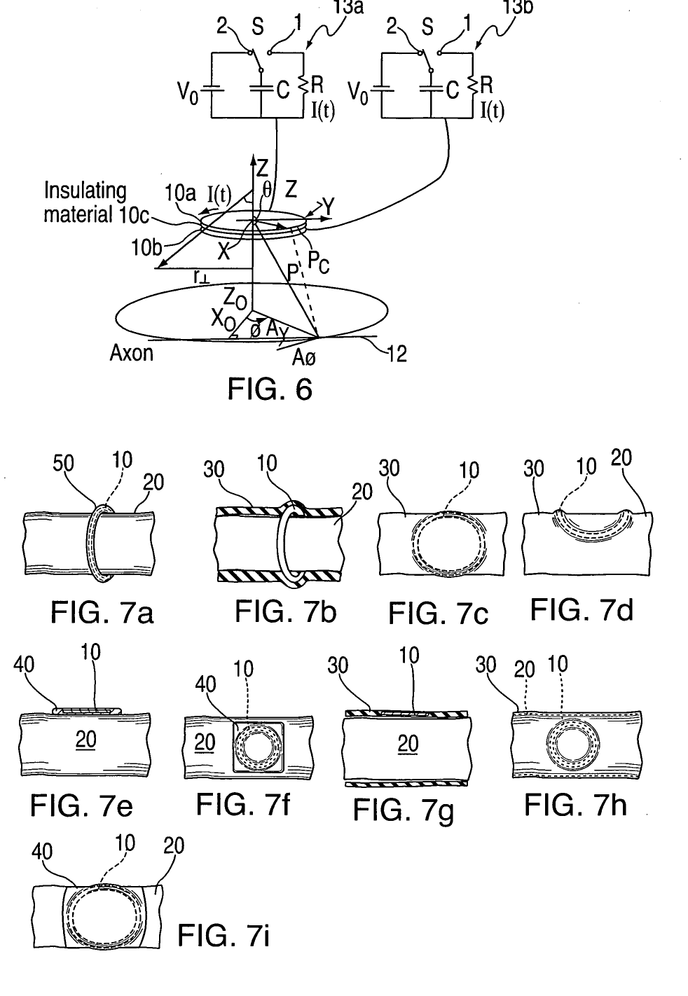

FIG. 6 illustrates such an arrangement. As shown, one coil 10a is positioned directly on top of the other coil 10b with insulating material 10c positioned between the two to prevent direct electrical contact. For the purpose of the rest of this document, the conglomerate of the two proximate coils will be referred to by the number 10.

In addition to the proximate coils 10, there is a high speed discharge circuit which will short circuit the capacitor in the RLC circuit 13a and 13b and, thus, bring the circuit current down to zero almost instantaneously. The two circuits 13a and 13b are timed so that the second circuit is pulsed with a DC discharge 50 microseconds after the first circuit was pulsed. Ten microseconds later, the first circuit is short circuited so that the current in the coil drops to zero.

Fifty microseconds later, the first circuit receives its next DC pulse. Ten microseconds later the second circuit is short-circuited. This cycle is repeated continuously so that there is always one coil with a large enough value of dI/dt to produce the requisite externally induced transmembrane potential in a continuous fashion.

Turning now to FIGS. 7a-7i, several different configurations of the present invention are illustrated.

FIGS. 7a and 7b depict an arrangement of coil 10 of the present invention in which coil 10 completely encircles penis 20. If desired, coil 10 can be housed within ring 50, as shown in FIG. 7a, or coil 10 can be housed within prophylactic 30, as shown in FIG. 7b.

FIG. 7c is a top view of an arrangement of coil 10 housed within prophylactic 30 and half-wrapped around penis 20, while FIG. 7d shows a side view of the FIG. 7c.

FIG. 7e is a side view of an arrangement of coil 10 in which coil 10 is housed within patch 40, and patch 40 is worn on the outside of penis 20, while FIG. 7f shows a top view of FIG. 7e.

FIG. 7g is a side view of an arrangement of coil 10 in which coil 10 is housed within prophylactic 30 and coil 10 is worn on the surface of penis 20, while FIG. 7h shows a top view of FIG. 7g.

FIG. 7i depicts an arrangement of the present invention in which coil 10 is mounted within patch 40 and patch 40 is half-wrapped on the outside of penis 20.

It will be understood that the claims are intended to cover all changes and modifications of the preferred embodiments of the invention herein chosen for the purpose of illustration which do not constitute a departure from the spirit and scope of the invention.

What is claimed is:

1. A method for stimulating a cavernous nerve in a patient comprising the steps of:
creating a time varying magnetic field with a device positioned completely external to said patient, said time varying magnetic field resulting in an electric current in said cavernous nerve, for as long as required, said electric current causing the propagation of a neural impulse in said neural tissue thereby resulting in an erection.

2. The method of claim 1 wherein said device comprises a coil of wire which can produce a time varying magnetic field.

3. The method of claim 2 wherein said coil of wire is housed within a housing so as to insulate said coil of wire.

4. The method of claim 1 wherein said device comprises a resistor, a capacitor and a coil of wire in series.

5. The method of claim 4 wherein said capacitor is discharged through said coil of wire so as to form a time varying magnetic field which in turn creates said electric field.

6. The method of claim 2 wherein said coil of wire is circular in shape and has about 7 to about 10 turns, and said coil of wire has a diameter of about 3 to about 7 cm.

7. The method of claim 1 wherein a time varying current passes through said device and said time varying current increases from 0 to about 6000 amps in 60 microseconds.

8. The method of claim 4 wherein said coil of wire has a resistance (R) of about 0.1 to about 0.5 ohms and an inductance (L) of about 10 to about 90 microhenries.

9. An electrical device for producing a nerve impulse in a cavernous nerve so as to produce an erection, said device comprising:
a RLC circuit comprising a resistor, a coil of wire, and a capacitor, wherein said coil of wire is positioned so as to situate said coil in close proximity to said cavernous nerve;
a housing for insulating said coil of wire.

10. The electrical device of claim 9 wherein said housing is a ring, a patch, or a prophylactic; and said housing is adapted to be worn on the outside of a penis.

11. The electrical device of claim 9 wherein said coil of wire completely encircles a penis.

12. The electrical device of claim 9 wherein said coil of wire is in a plane substantially perpendicular to a plane of said cavernous nerve.

13. The electrical device of claim 9 wherein said coil of wire is in a plane substantially parallel to a plane of said cavernous nerve.

14. The electrical device of claim 9 wherein said coil of wire is half-wrapped around a penis.

15. The electrical device of claim 9 wherein current produces an electrical field which create a triphasic potential within the cavernous nerve, said triphasic potential consisting of a virtual cathode surrounded by a virtual anode on either side of said virtual cathode.

16. The electrical device of claim 9 wherein the RLC circuit creates a time varying magnetic field, said time varying magnetic field resulting in an electric current in said cavernous nerve, for as long as required, said electric current causing the propagation of a neural impulse in said neural tissue thereby resulting in an erection.

17. The electrical device of claim 9 wherein said coil of wire is circular in shape and has about 7 to about 10 turns, and the coil has a diameter of about 3 to about 7 cm.

18. The electrical device of claim 9 wherein a time varying current passes through said RLC circuit and said time varying current increases from 0 to about 6000 amps in 60 microseconds.

19. The electrical device of claim 9 wherein said coil of wire has a resistance (R) of about 0.1 to about 0.5 ohms and an inductance (L) of about 10 to about 90 microhenries.
